(19) United States
(12) Patent Application Publication
(10) Pub. No.: US 2006/0064070 A1
(43) Pub. Date: Mar. 23, 2006

(54) Methods and devices for sterile field transfer

(76) Inventor: Jeffrey Martin, Memphis, TN (US)

Correspondence Address: CHIEF PATENT COUNSEL;SMITH & NEPHEW, INC., 1450 BROOKS ROAD, MEMPHIS, TN, 38116, US

(21) Appl. No.: 11/220,834

(22) Filed: Sep. 7, 2005

Related U.S. Application Data
(60) Provisional application No. 60/607,631, filed on Sep. 7, 2004.

(51) Int. Cl. A61B 19/00

(52) U.S. Cl. 604/403

(57) ABSTRACT

Devices and methods for facilitating the transfer of at least a component of a material into a sterile vessel. Such devices and methods may utilize a housing at least partially enclosing the sterile vessel to facilitate maintaining the sterility of the vessel before and during the transfer of the at least a component of a material into the sterile vessel.

### RELATED APPLICATIONS

This application claims the benefit of U.S. Provisional Patent Application Ser. No. 60/607,631, entitled “Sterile Field Transfer Method and Design” filed Sep. 7, 2004, the entire contents of which are hereby incorporated by this reference.

### RELATED FIELDS

Embodiments of the present invention relate to methods and devices for transferring at least a component of a material into a sterile vessel for use in a sterile field.

### BACKGROUND

It is often necessary to ensure that items used in a sterile field, such as an operating room, research & development facility, or other sterile environment, are themselves sterile. Instruments, devices, containers, implants and other items intended for use in the sterile field may require sterilization and may need to be maintained in a sterile condition. Items that are non-sterile, or that potentially have contacted other non-sterile items or non-sterile persons (such as doctors, technicians or nurses who have come into contact with non-sterile items), may be unacceptable for use in the sterile field. Accordingly, doctors, nurses, technicians and other individuals working in or around sterile fields must follow strict guidelines and procedures for ensuring the sterility of items brought into and/or used in the sterile field. Some of the current sterilization procedures and guidelines, however, are undesirable, burdensome and inefficient.

For example, typical procedures for collecting and transporting blood platelets into a sterile field may be undesirable, as those procedures may require the transfer of the platelets between a number of different containers and devices. Typically, the nurse, technician, doctor or other individual collects a blood sample in a syringe. Next, he or she transfers the blood into a centrifuge. The centrifuge processes the blood to concentrate the blood platelets. Next, the concentrated blood platelets are collected into another syringe and subsequently transferred into a sterile cup. The sterile cup is finally brought into the sterile field where it can be drawn into a sterile syringe. Such a technique is undesirable, as it requires the blood and blood platelets to be transferred multiple times from vessel to vessel in a manner that is inefficient and subject to spillage or even accidental contamination.

Other procedures, techniques and guidelines for ensuring the sterility of items used in sterile fields may also be undesirable, burdensome and inefficient.

### SUMMARY

Embodiments of the present invention provide safe and efficient methods and devices for transferring a material or a component of a material from a non-sterile vessel or potentially non-sterile vessel into a sterile vessel, while maintaining the sterility of the sterile vessel. Embodiments of the present invention may be suitable for transferring a wide variety of materials or material components into a sterile vessel, including, but not limited to, blood, blood components (such as platelets or blood plasma), bone cement, ceramics, allogenic bone, autologous bone, bone marrow, other fluids, polymers, composite materials, nanomaterials, or any other material or material component that is intended for use in a sterile field. Devices according to various embodiments of the present invention may also process the materials or material components introduced into the device prior to or during transfer into the sterile vessel. Exemplary processes include, but are not limited to, filtration to separate larger components of a material from smaller components (e.g, filtering blood to separate the platelets from other blood components), mixing materials together, separating materials, reacting various materials together, instrument fabrication, implant fabrication, sterilization or any other process that may be done to a material or a material component prior to use in a sterile field. In other embodiments, devices of the present invention do not process the materials introduced into them at all, but merely facilitate the material transfer into the sterile vessel without affecting the sterility of the sterile vessel.

Devices of the present invention may include a sterile vessel and a housing at least partially enclosing the sterile vessel. The device housing may enclose the sterile vessel to protect the vessel from direct contact with potentially non-sterile persons and items, which could potentially affect the sterility of the sterile vessel and prevent use of the vessel in the sterile field. In some embodiments, the housing may include a cover that limits access to the sterile vessel until the cover is removed or opened. In other embodiments, such a cover is unnecessary.

Devices according to the present invention may also include a material input accessible from outside the housing. The material input may facilitate the introduction of a material into the device, which eventually may be transferred by the device (or a component of the material may be transferred by the device) into the sterile vessel. In some embodiments, the material input interacts with a non-sterile syringe or other vessel external to the device. In some embodiments, the material input is associated with a conduit within the device that conveys the material to various internal components of the device (such as a filter) and to the sterile vessel. In other embodiments, the conduit is integral with the material input and conveys the material directly into the sterile vessel.

Some embodiments of the present invention may also include internal components, such as filters or other components, for processing the inputted material. In some embodiments, the device may also include additional material inputs and/or internal components for introducing and/or processing additional materials in the device. The additional materials introduced may facilitate various processes of the device and/or may facilitate the movement of other materials (or components thereof) within and between various internal components of the device. In other embodiments, the device does not include such components and merely conveys the material directly into the sterile vessel.

In some embodiments, the device is disposed of after use and is made with relatively inexpensive materials.

### BRIEF DESCRIPTION OF FIGURES

FIG. 1 is a schematic of a device according to a first embodiment of the present invention.

FIG. 2 is a perspective view of a device according to another embodiment of the present invention, shown without the device's housing.

FIG. 3 is a perspective view of a device according to another embodiment of the present invention, shown without the device's housing.

FIG. 4 is a perspective view of a device according to another embodiment of the present invention, shown without the device's housing.

FIG. 5(a) is a perspective view of the exterior of a device according to another embodiment of the present invention.

FIG. 5(b) is a side view of the device of FIG. 5(a).

FIG. 6(a) is a perspective view of the exterior of a device according to another embodiment of the present invention.

FIG. 6(b) is a side view of the device of FIG. 6(a).

FIG. 6(c) is a rear perspective view of the device of FIG. 6(a).

FIG. 7(a) is a perspective view of the exterior of a device according to another embodiment of the present invention.

FIG. 7(b) is a side view of the device of FIG. 7(a).

FIG. 8(a) is a perspective view of the exterior of a device according to another embodiment of the present invention.

FIG. 8(b) is a side view of the device of FIG. 8(a).

FIG. 9(a) is a perspective view of the exterior of a device according to another embodiment of the present invention.

FIG. 9(b) is a side view of the device of FIG. 9(a).

### DETAILED DESCRIPTION OF FIGURES

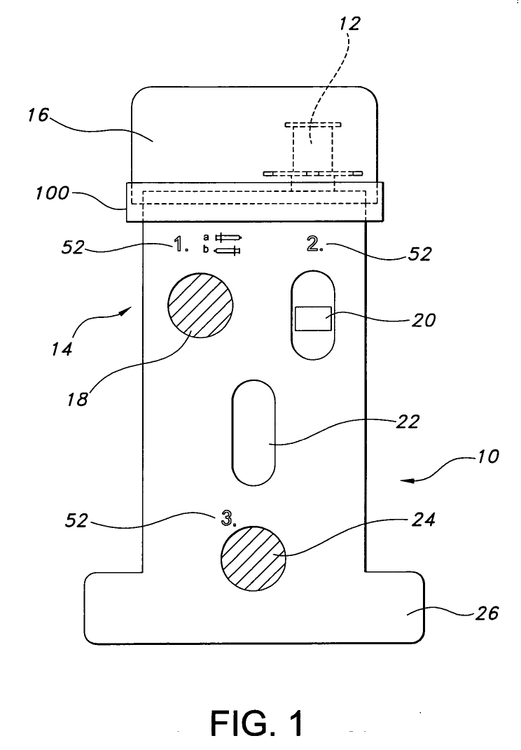

FIG. 1 shows a device 10 in accordance with embodiments of the present invention. In the configuration shown in FIG. 1, device 10 includes a sterile vessel 12, a housing 14 (which includes a cover 16), a material input 18, an actuator 20, a window 22, a second material input 24, a base 26, and a seal 100. In other embodiments, some or all of these features are unnecessary and/or are present in different configurations, positions, orientations, shapes and sizes.

The sterile vessel 12 shown in FIG. 1 is a syringe, however, sterile vessel may be any vessel suitable for receiving materials or components of materials, including test tubes, beakers, vials, IV bags, sample holders, pails, cups, bowls, trays, or gloved hands. Sterile vessel 12 may be sterilized prior to installation in device 10 or sterilized along with the rest of device 10 or components of device 10. Sterile vessel 12 (and other components of device 10 if desired or required) may be sterilized in any suitable procedure or technique, including, but not limited to, gas sterilization or gamma radiation. In other embodiments, it is unnecessary to subject sterile vessel 12 and/or other components of device 10 to a sterilization procedure and/or the sterile vessel may have been maintained in a sterile condition since its manufacture.

The housing 14 shown in FIG. 1 encloses sterile vessel 12. Housing 14 is preferably formed from a plastic, such as polycarbonate, however, housing 14 may be formed from other materials if desired. In some embodiments, it is desirable to form housing 14 (and other components of device 10) from relatively inexpensive materials, as device 10 may be intended to be disposable or intended for “one time use” in some configurations.

Because the housing 14 shown in FIG. 1 encloses sterile vessel 12, the sterility of vessel 12 may be preserved even if device 10 is used in non-sterile fields or comes into contact with non-sterile persons or things. In the embodiment of FIG. 1, the housing 14 (which includes the cover 16 in the configuration shown in FIG. 1) preserves the sterility of vessel 12 until such time as cover 16 is removed or repositioned to allow access to sterile vessel 12 by a sterile nurse, doctor, technician or other individual for use in a sterile field. In some embodiments, a non-sterile individual will remove or reposition the cover 16 and a sterile individual will remove the sterile vessel 12 such that the vessel 12 does not contact non-sterile persons or things and such that the sterile person does not contact the potentially non-sterile housing 14 of device 10. In other embodiments, device 10 may be configured to automatically move the cover 16 or otherwise facilitate access to sterile vessel 12 after the occurrence of one or more events. For instance, in some embodiments, the transfer or completion of transfer of a material into sterile vessel 12 may trigger the device 10 to automatically move the cover 16 or otherwise facilitate accessing vessel 12.

In some embodiments, one person will transport the device 10 into the sterile field and the sterile person will remove the sterile vessel 12. In other embodiments, the sterile person will remove the sterile vessel 12 from the device 10 outside of the sterile field, and then enter the sterile field with the sterile vessel 12.

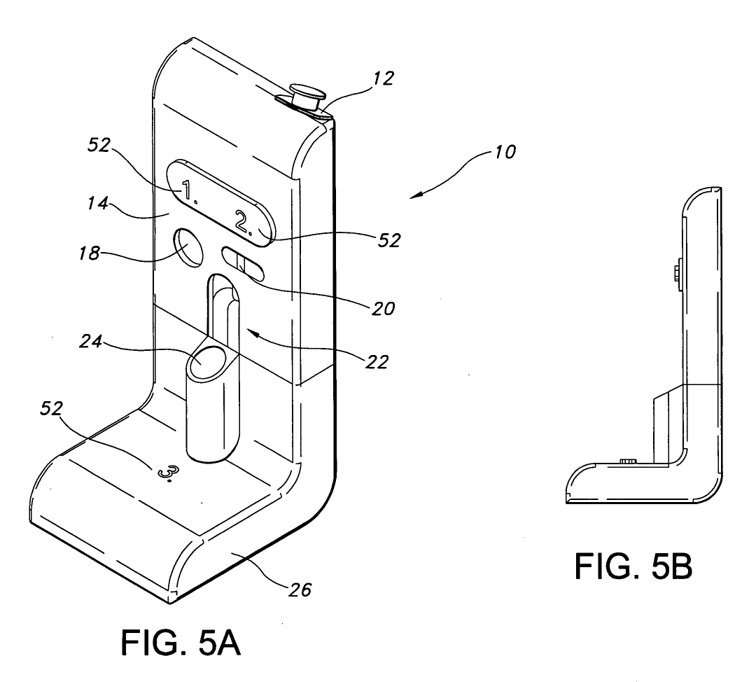

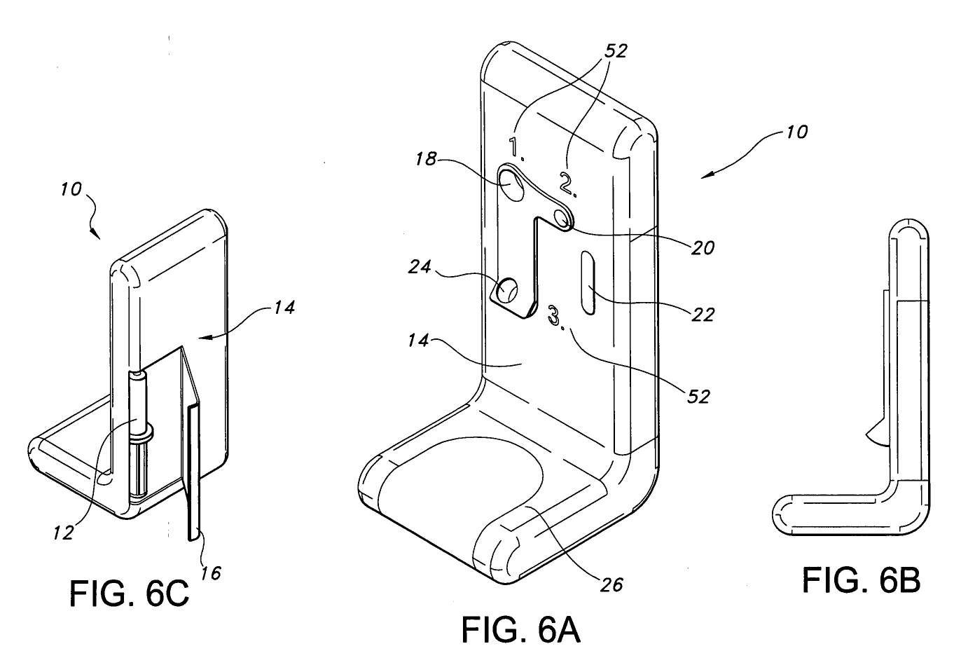

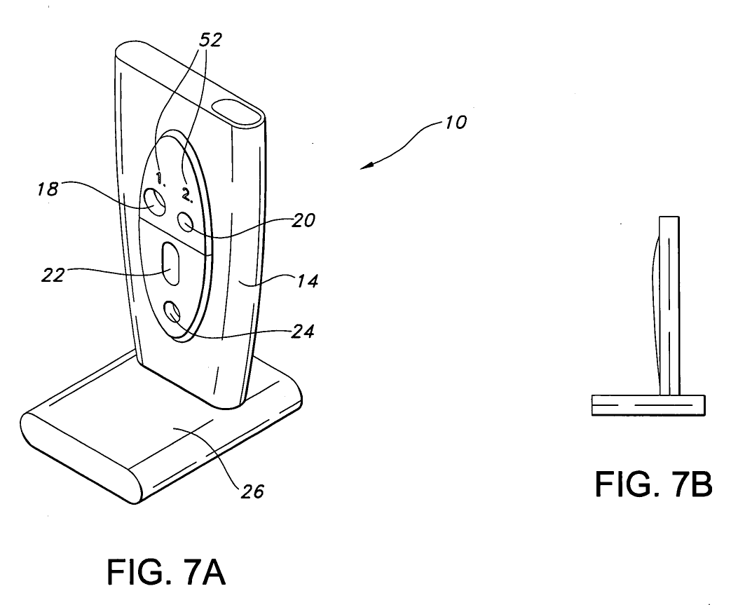

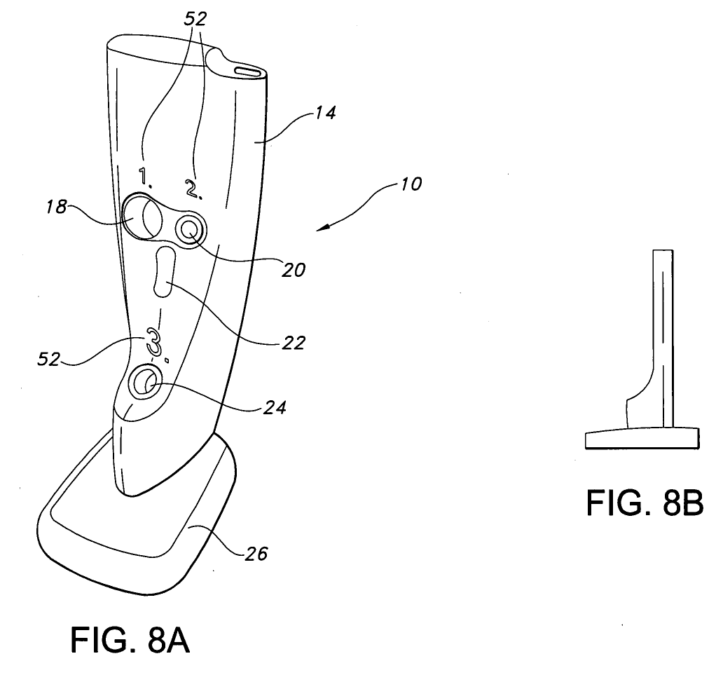

As shown in FIG. 1, cover 16 is a removable lid. In other embodiments, a cover 16 similar to the one shown in FIG. 1 can be used in conjunction with the devices 10 shown in FIGS. 5(a), 5(b), 7(a), 7(b), 8(a), 8(b), 9(a) and 9(b), although the devices 10 shown in these Figures may not necessarily require such a cover in all embodiments. In still other embodiments, such as shown in FIG. 6(c), cover 16 may be a hatch mounted to device 10 in a rotating fashion.

As shown in FIG. 1, cover 16 includes a removable or breakable seal 100 for indicating whether sterile vessel 12 has been previously accessed or tampered with (indicating a potential loss of sterility). A user (in some embodiments, the non-sterile nurse, doctor, technician or other non-sterile individual) may break or remove the seal 100 and remove or reposition the cover 16 to allow access to sterile vessel 12. The seal 100 may be any desired mechanism, structure, device or item for sealing cover 16 onto device 10, including frangible tabs, adhesive tape, gaskets, tamper indicating mechanisms, or any other suitable structure or mechanism for sealing cover 16 prior to removal or repositioning. FIG. 1 shows seal 100 as an adhesive tape at least partially encircling the connection between cover 16 and the rest of housing 14. In still other embodiments, a separate mechanism, structure, device or item is not necessary to seal cover 16 to the rest of housing 14 and the cover is formed integrally with the rest of housing 14. In such embodiments, cover 16 may be punctured, broken or otherwise destroyed once it is desired to access sterile vessel 12. In other embodiments, a seal is unnecessary.

In yet other embodiments, cover 16 is unnecessary. For example, the housing 14 may include an aperture in which sterile vessel 12 is located. Sterile vessel 12 may be positioned deep enough within the housing 14 such that non-sterile persons or things cannot easily contact sterile vessel 12. In such embodiments, device 10 may include a slider or other mechanism for facilitating access to the sterile vessel 12 at the appropriate time. For example, in some embodiments, actuation of the slider may raise sterile vessel 12 into an accessible position. In other embodiments, as the sterile vessel 12 fills with material (described further below), a plunger of the vessel may extend outside of the housing, allowing a sterile nurse, doctor or technician to grasp the end of the plunger and remove sterile vessel 12 from device 10. In some embodiments, the housing 14 may include a plastic film over the aperture or other portions of the housing 14, such that breaking the film allows access to the vessel 12.

The device 10 of FIG. 1 also includes a material input 18. As discussed above, material may be any material that a user desires to transfer into a sterile vessel 12, including blood, blood components (such as platelets or blood plasma), bone cement, ceramics, allogenic bone, autologous bone, bone marrow, other fluids, or any other material. Material input 18 may be configured to receive and convey any desired material into device 10. In one embodiment, material input 18 includes an aperture that corresponds to the size and shape of a syringe tip such that material input 18 receives the tip in a manner that substantially prevents leakage. In some embodiments, material input 18 is an aperture in housing 14 that allows the syringe or other vessel to interact with internal components such that the syringe can introduce material into device 10. Material input 18 may also include a gasket or other appropriate structures or mechanisms for lessening the chance of leakage. In other embodiments, material input 18 does not interact with a syringe to receive material, but is sized, shaped and positioned for material to be poured directly into material input 18. For example, in some embodiments, material input 18 may be a funnel.

In still other embodiments, material input 18 may comprise other structures or mechanisms for facilitating the input of material into device 10. Additionally, material input 18 may or may not include a check valve or other appropriate structures and mechanisms for preventing material from flowing back out of material input. In some embodiments, material input 18 may include a filter, such as a screen, or other structure for separating impurities or other things from material during input into device 10.

The device 10 shown in FIG. 1 also includes an actuator 20. The actuator 20 may be a slide (such as shown in FIGS. 1 and 5(a)), a knob, a button (such as shown in FIGS. 6(a), 7(a), 8(a) and 9(a)) or other suitable structure or mechanism for commencing processes or otherwise affecting structures inside device 10, some examples of which are discussed further below. Although FIG. 1 shows device 10 as only including a single actuator 20, devices 10 in accordance with embodiments of this invention may include any number of actuators 20 for controlling or affecting various internal components of device 10. In still other embodiments, an actuator 20 is unnecessary.

The device 10 of FIG. 1 also includes a window 22. In some embodiments, window 22 may be a transparent or semi-transparent window. Window 22 may allow viewing of one or more of the internal components of device 10 and/or the progress of the inputted material through the same. As shown in FIGS. 1, 5(a), 6(a), 7(a), 8(a) and 9(a), the size, shape, number and position of windows 22 may be varied to facilitate viewing various interior portions and/or components of device 10. In other embodiments, window 22 is unnecessary and/or other features of device 10 indicate the progress of the inputted material through device 10. For example, sterile vessel 12 may include a plunger that extends as material fills the vessel 12, which may roughly indicate the amount of material that has moved into the vessel. In some embodiments, device 10 does not include any features or components for indicating the progress of material through device 10.

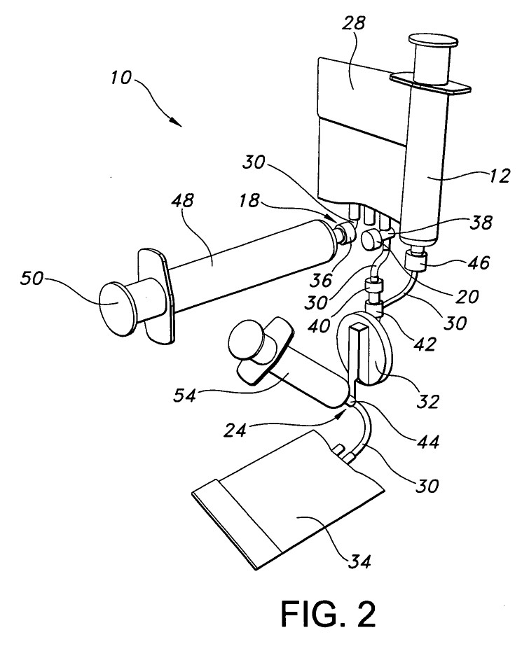

In the configuration shown in FIG. 1, device 10 includes a second material input 24. Like embodiments of material input 18 described above, second material input 24 may interact with the tip of a syringe 54 (shown in FIGS. 2-4) to receive and convey a material into device 10 or may include other structures and mechanisms as discussed above for material input 18. Second material input 24 is also discussed further below.

In the configuration shown in FIG. 1, device 10 also includes a base 26. Base 26 may maintain the device 10 in an upright orientation when placed on a surface. Moreover, if desired, a user may push down on base 26 to stabilize device 10 during use. As shown in FIGS. 5(a)-9(b), base 26 may be formed in a wide variety of shapes, sizes and configurations.

The devices 10 shown in FIGS. 1-9(b) are configured to separate the platelet component of a blood material and transfer those platelets into sterile vessel 12. Devices 10 in accordance with other embodiments of the present invention, however, may be configured to perform a wide variety of processes on a wide variety of materials. Exemplary processes include, but are not limited to, filtering, mixing, separating, reacting, fabrication, or sterilization. In still other embodiments, device 10 does not process the inputted material at all, but merely transfers the material into sterile vessel 12. Exemplary materials useable in conjunction with devices 10 in accordance with the present invention are discussed above.

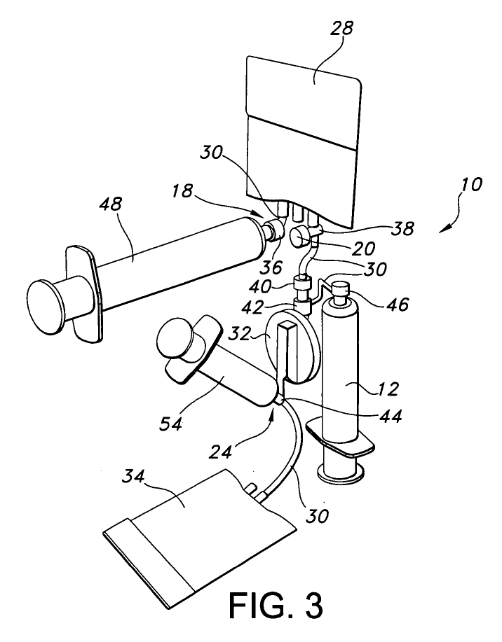

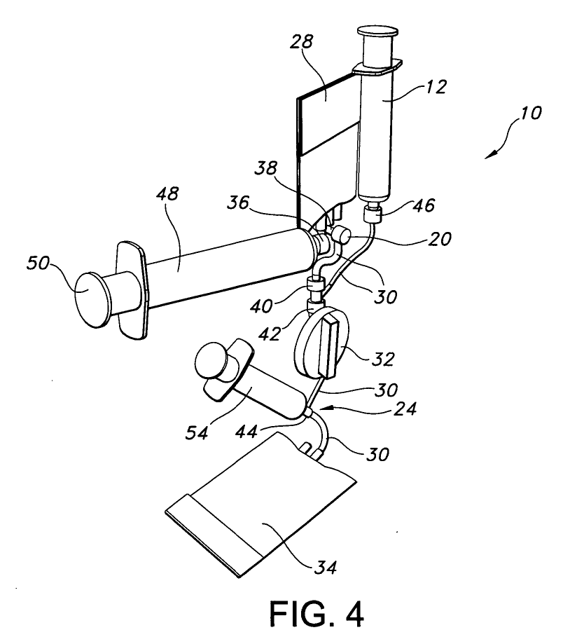

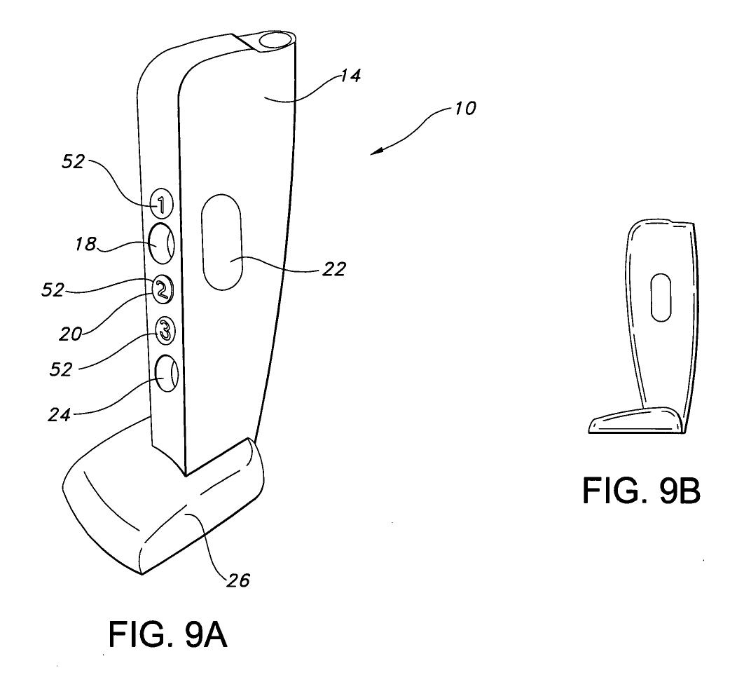

FIGS. 2-4 illustrate various configurations of internal components of device 10 for filtering and transferring the blood platelet component of an inputted blood material into sterile vessel 12. As shown in FIGS. 2-4, the locations and orientations of the internal components can be varied based on the shape and size of housing 14 (e.g., the configuration of FIG. 2 may be suitable for the housings 14 of FIGS. 5(a), 5(b), 7(a), 7(b), 8(a) and 8(b), the configuration of FIG. 3 may be suitable for the housing 14 of FIGS. 6(a), 6(b) and 6(c), and the configuration of FIG. 4 may be suitable for the housing 14 of FIGS. 9(a) and 9(b)). In the configurations shown in FIGS. 2-4, device 10 includes the following internal components: material reservoir 28, conduits 30, filter 32, waste reservoir 34, and various valves 36, 38, 40, 42, 44 and 46. Device 10 configurations in accordance with other embodiments of the present invention may exclude some or all of these components and/or include other components.

In the configurations of FIGS. 2-4, introduction of blood material into device 10 via material input 18 (as discussed above) directs the material into material reservoir 28. Syringe 48 interacts with material input 18 to facilitate the introduction of blood material into device 10 in the embodiments of FIGS. 2-4, however, other vessels, devices or methods can also be used to introduce a material into devices 10 in accordance with the present invention (some examples of which, but not all, are discussed above). In the configurations of FIGS. 2-4, depressing a plunger 50 associated with the syringe 48 may increase the pressure on blood material within syringe 48, causing it to flow through material input 18 into material reservoir 28.

FIGS. 2-4 show a conduit 30 for directing material from syringe 48 into material reservoir 28. Other portions of conduit 30 shown in FIGS. 2-4 direct the movement of materials and components of materials between other components of device 10. In the embodiments shown in FIGS. 2-4, conduit 30 is a plastic tubing, although in other embodiments, conduit 30 may be any suitable device, structure or mechanism for containing and/or facilitating the movement of material between various components of device 10. In some embodiments, integral portions of material input 18 are a conduit for directing material into material reservoir 28 or other components of device 10.

The devices 10 of FIGS. 2-4 include a check valve 36 positioned where material is input into material reservoir 28. Check valve 36 may regulate the flow of material such that material can enter material reservoir 28 through check valve 36, but may not exit material reservoir 28 through check valve 36. Check valve 36 may prevent leakage of material from device 10 once the material has entered material reservoir 28. In other embodiments, check valve 36 is unnecessary. For example, material reservoir 28 may be a sufficient size to contain the material from syringe 48 and/or may include the material input at an upper portion of the reservoir to lessen the chance of undesired leakage.

In still other embodiments, material reservoir 28 is unnecessary and the introduced blood or other material passes directly to other internal components of the device 10 from material input 18 and/or check valve 36.

FIGS. 2-4 show that material reservoir 28 includes another valve 38 for regulating the movement of material out of the material reservoir 28. Valve 38 may be operatively associated with actuator 20 shown in FIG. 1. Actuation of actuator 20 may open and/or close valve 38. Opening valve 38 may allow material to move from material reservoir 28 to other downstream components of device 10. In the embodiments of FIGS. 2-4, gravity facilitates moving the material out of material reservoir 28 once valve 38 is opened by actuator 20. In other embodiments, actuator 20 is unnecessary and valve 38 may automatically open once a sufficient amount of material builds up in material reservoir 28. In still other embodiments, valve 38 is unnecessary and material can move directly from material reservoir 28 to down-stream components of device 10.

In the configuration shown in FIGS. 2-4, once valve 38 opens, material may move from material reservoir 28 into filter 32. In the configurations of FIGS. 2-4, wherein the material is a blood material, filter 32 may process the blood to separate platelets from other components of the blood. Filter 32 may include a screen having a plurality of apertures that allow smaller components of the blood material to pass through, while retaining the platelets that are too large to pass through the screen. Screens having different sized apertures may be used depending on the size of the components to be retained by filter 32. The components of material that pass through the screen may subsequently be directed by diverter valve 44 (discussed further below) into waste reservoir 34. In other embodiments, filter 32 may include other structures or mechanisms for separating material components from one another.

In other embodiments, the material components that pass through the filter 32 screen may be reserved while the components that are too large to pass through the screen may eventually be directed into a waste reservoir 34 or may simply remain within the filter 32. In still other embodiments, both the material passing through the filter 32 and the material retained within the filter 32 may be reserved and collected into various vessels. In such embodiments, device 10 may include two sterile vessels to collect both material components.

In the embodiments shown in FIGS. 2-4, a back-flush procedure may be used to transfer the material components retained in filter 32 into sterile vessel 12. During the back-flush procedure, a back-flush material may be introduced through a second material input 24 and travel through the filter 32 screen in the opposite direction that the blood or other material originally traveled through the filter 32. As the back-flush material travels through the filter 32, it may pick up the platelets or other material components reserved by the filter 32 and carry those reserved components out of the filter 32. The diverter valve 42 (discussed further below) shown in FIGS. 2-4 may direct the back flush material carrying the reserved component into the sterile vessel. Back-flush material may be any material suitable for carrying the platelets or other material component reserved by filter 32 out of filter 32 and into sterile vessel 12, including, but not limited to, any fluid. Back-flush material may be introduced into device 10 using a syringe 54 or other suitable vessel, device or method. In other embodiments, back-flush procedures and/or back-flush materials are unnecessary.

The diverter valves 42 and 44 shown in FIGS. 2-4 regulate the direction and sources/destinations of flow through the filter 32. During the filtration process, diverter valve 42 may be positioned to allow material to pass from material reservoir 28 into the filter 32 and diverter valve 44 may be positioned to allow the material components passing through filter 32 to move into waste reservoir 24. During the back-flush procedure, diverter valve 44 may be positioned allow the back-flush material introduced through second material input 24 to pass into filter 32 and diverter valve 42 may be positioned to allow the back-flush material and filtered component to pass into sterile vessel 12. In the embodiments shown in FIGS. 2-4, device 10 also includes check valve 40, which may prevent back-flush material and the filtered components from entering material reservoir 28.

The diverter valves 42 and 44 shown in FIGS. 2-4 are configured to automatically switch depending on the direction of flow through the valve body, thereby directing the material or material component to the proper component. In other embodiments, diverter valves 42 and 44 are manually switched using actuators accessible from outside of housing 14. In still other embodiments, diverter valves 42 and 44 and/or check valve 40 may be unnecessary and additional conduit or other structures may be utilized to direct the material flow through the components of device 10 in the proper directions and to the proper destinations.

In the embodiments shown in FIGS. 1 and 6(a)-8(a), device 10 includes indicia 52 for indicating the order of the various steps for processing and/or collecting blood platelets (or other materials or components of materials) into sterile vessel 12.

In some embodiments, device 10 may be disposed of after the removal of sterile vessel 12.

Modifications, additions and deletions may be made to the embodiments described above and shown in the accompanying figures without departing from the scope or spirit of the present invention.

1. A device for facilitating the transfer of at least a component of a material into a sterile vessel, the device comprising:
(a) a sterile vessel;
(b) a housing at least partially enclosing the sterile vessel;
(c) a material input for facilitating the input of a material into the device, the material input accessible from outside the housing; and
(d) a conduit for guiding movement of at least a component of the material from the material input to the sterile vessel.

2. The device for facilitating the transfer of at least a component of a material into a sterile vessel of claim 1, wherein the housing comprises a cover and wherein moving the cover facilitates accessing the sterile vessel.

3. The device for facilitating the transfer of at least a component of a material into a sterile vessel of claim 2, wherein the cover is at least temporarily sealed on the device.

4. The device for facilitating the transfer of at least a component of a material into a sterile vessel of claim 2, wherein the cover comprises a removable cap or a moveable hatch.

5. The device for facilitating the transfer of at least a component of a material into a sterile vessel of claim 4, wherein the housing comprises an at least semi-transparent window.

6. The device for facilitating the transfer of at least a component of a material into a sterile vessel of claim 2, wherein the housing further comprises at least one actuator for actuating at least one valve, the at least one valve for at least partially regulating the movement of the at least a component of the material through the conduit.

7. The device for facilitating the transfer of at least a component of a material into a sterile vessel of claim 1, further comprising a reservoir associated with the conduit.

8. The device for facilitating the transfer of at least a component of a material into a sterile vessel of claim 7, wherein the reservoir is associated with the material input and receives material from the material input.

9. The device for facilitating the transfer of at least a component of a material into a sterile vessel of claim 1, further comprising a filter for separating the component from a second component of the material.

10. The device for facilitating the transfer of at least a component of a material into a sterile vessel of claim 9, wherein the conduit directs the component into the sterile vessel.

11. The device for facilitating the transfer of at least a component of a material into a sterile vessel of claim 10, further comprising a second material input for receiving a second material, wherein introduction of the second material into the device facilitates moving the component from the filter to the sterile vessel.

12. The device for facilitating the transfer of at least a component of a material into a sterile vessel of claim 10, further comprising a second reservoir for collecting the second component from the filter.

13. A method for facilitating the transfer of at least a component of a material into a sterile field, comprising:
(a) collecting a material;
(b) transferring the material into a device, the device comprising:
(i) a sterile vessel; and
(ii) a housing at least partially enclosing the sterile vessel;
(c) transporting the sterile vessel into a sterile field; and
(d) removing the sterile vessel from the device.

14. The method for facilitating the transfer of at least a component of a material into a sterile field of claim 13, wherein the sterile vessel is transported into the sterile field prior to removing the sterile vessel from the device.

15. The method for facilitating the transfer of at least a component of a material into a sterile field of claim 13, wherein the sterile vessel is removed from the device prior to transporting the sterile vessel into the sterile field.

16. The method for facilitating the transfer of at least a component of a material into a sterile field of claim 13, wherein removing the sterile vessel from the device further comprises moving a cover of the housing of the device to access the sterile vessel.

17. The method for facilitating the transfer of at least a component of a material into a sterile field of claim 16, wherein moving the cover comprises removing the cover from the device.

18. The method for facilitating the transfer of at least a component of a material into a sterile field of claim 16, wherein moving the cover comprises opening a hatch.

19. The method for facilitating the transfer of at least a component of a material into a sterile field of claim 13, further comprising filtering the material to separate the material into a first component and a second component.

20. The method for facilitating the transfer of at least a component of a material into a sterile field of claim 13, further comprising disposing of the device.
